(19) United States
(12) Patent Application Publication
(10) Pub. No.: US 2025/0262152 A1
(43) Pub. Date: Aug. 21, 2025

(54) DEVICES, SYSTEMS, KITS, AND METHODS FOR DRUG DELIVERY TO THE SPINAL CORD

(71) Applicant: Ohio State Innovation Foundation, Columbus, OH (US)

(72) Inventors: Bryan TILLMAN, Columbus, OH (US); Dahlia KENAWY, Columbus, OH (US)

(21) Appl. No.: 18/859,451

(22) PCT Filed: Apr. 24, 2023
(86) PCT No.: PCT/US2023/019685

§ 371 Date: Oct. 23, 2024

Related U.S. Application Data
(60) Provisional application No. 63/334,125, filed on Apr. 23, 2022.

(51) Int. Cl. A61K 9/00; A61K 31/417; A61K 45/06

(52) U.S. Cl. CPC A61K 9/0085; A61K 31/417; A61K 45/06

(57) ABSTRACT

Disclosed are devices, systems, methods and kits for delivering active agents to the spinal cord.

### CROSS-REFERENCE TO RELATED APPLICATIONS

This application claims benefit of U.S. Provisional Application No. 63/334,125, filed Apr. 23, 2022, which is hereby incorporated herein by reference in its entirety.

### TECHNICAL FIELD

This application relates generally to devices, systems, kits, and methods for regional drug delivery to the spinal cord.

### BACKGROUND

Spinal cord injury/degeneration remains one of the greatest challenges in modern medicine. Promising candidate therapies span a wide variety of cellular, molecular and even gene therapies, yet they all share a similar hurdle—it remains a challenge to effectively deliver these agents to the spinal cord. Many existing avenues for drug delivery require injection and/or surgery, both of which risk further iatrogenic damage of the spinal cord. Alternatively, intravascular drug delivery is systemic, diluting the drug into the entire circulation.

Accordingly, improved strategies for the delivery of active agents to the spinal cord are needed.

### SUMMARY

Effective drug delivery to the spinal cord is attractive for a number of reasons. First, reversal of spinal cord injury remains a critical goal for medical researchers. However, regardless of the therapy administered, any active agents must be effectively delivered across the blood brain barrier (BBB) to reach the spinal cord. The use of surgery or injection to deliver an active agent can cause further injury whether by the surgical tool or needle, or damaging compression from the injected agent. Furthermore, the agent may not permeate the entire spinal cord. Similar challenges face treatment of neurodegenerative disorders.

Second, unlike cancers in other organs, cancers of the spinal cord cannot undergo any sort of resection without tremendous disability, leaving patients to the hazards and complications of radiation or to systemic chemotherapy.

In both of these scenarios, whether the therapy is an antibody, gene therapy, immunomodulatory agents, chemical, or chemotherapeutic, the vascular system offers perfusion to the entirety of the spinal cord. Nevertheless, by injection of these agents systemically (i.e., intravenously), the agent is diluted into a large volume of distribution. This in turn leads to toxicity to non-target tissues, excretion or inactivation by the liver and kidneys as well as increased cost since more of the drug is required to account for this dilutional/excretory effect.

A second, but certainly not less significant barrier of intravascular drug delivery to the spinal cord is the blood brain barrier which prevents all but a few small molecules from passing from the circulation to the spinal cord. As a result, the blood brain barrier (BBB) is prohibitive to delivery of almost every therapy to the spinal cord. A few compounds, of which histamine is the most potent, can disrupt the blood brain barrier temporarily. Since histamine is the primary mediator of allergy symptoms, it follows that giving a large systemic dose is certainly unwise.

Provided herein are methods for the delivery of active agents to the spinal cord that involve the local administration of a portal agent (e.g., histamine) and an active agent to a region of aortic branches that perfuse the spinal cord. The portal agent can increase permeability of the blood brain barrier, allowing for the active agent, which otherwise may not be BBB-permeable, to cross the BBB.

In some cases, local administration can be performed using a device, such as a retrievable stentgraft (e.g., a nitinol frame covered in polymer) that is dumbbell shaped. The larger top and bottom ends can form a vascular seal while the narrow middle portion leaves an outer chamber between the outside of the center lumen and the vascular wall. A small perfusion lumen is attached to the outer chamber. This perfusion lumen can be used to deliver the portal agent and active agent to the outer chamber and any branches off this isolated vessel segment (e.g., aortic branches that perfuse the spinal cord).

These features offer a myriad of therapeutic options. As several levels of spinal cord branches are isolated drug delivery can be restricted to the branches serving the spinal cord. As a result, a smaller overall dose of an active agent can be delivered to the patient, but at a higher effective dose at the spinal cord level than if given systemically. This can in turn reduce toxicity of the active agent as well as the volume/cost of the agent.

With regards to the blood brain barrier a dual chamber stent again can isolate the spinal perfusion, allowing a small “pulse” of a portal agent to be administered to increase the permeability of the blood brain barrier at exact region where the active agent is going to be subsequently delivered.

By isolating regions of spinal cord perfusion, the dual chamber stent allows smaller doses of both an agent to open the blood brain barrier and immediately thereafter, a therapeutic agent to be delivered to the spinal cord. This approach can minimize systemic contamination and ensure high dose therapy directly to the spinal cord. This can minimize toxicity to other tissues and loss of drug through “first pass kinetics” of the liver and kidneys.

The self expanding stentgraft can be composed of nitinol and polytetrafluoroethylene which are widely known among the industry for medical devices. The stent can be retrievable, allowing the stent to be rapidly recaptured, for example, by sliding a sheath over a fixed delivery wire on the stent, so that the stent can be removed from the aorta following administration of the active agent.

In other examples, the stent can have the perfusion lumen that is connected to a separate arterial line (to a contralateral femoral artery for instance) such that there remains continuous oxygenated perfusion to avoid spinal cord ischemia during prolonged drug therapy sessions.

Also provided herein are kits for delivering an active agent to the spinal cord of a patient. These kits can comprise a composition comprising an effective amount of a portal agent to disrupt the blood brain barrier of the patient; and an implantable device for localing administering the composition comprising the effective amount of the portal agent to a region of aortic branches that perfuse the spinal cord.

### DESCRIPTION OF DRAWINGS

FIG. 1 illustrates the use of a stentgraft to deliver agents to an isolated region of the vasculature. As shown in Panel A, a dual chamber stent can seal against the vessel wall at the top and bottom of the stentgraft, creating an isolated perfusion chamber (also referred to as the “outer chamber”). Normal blood flow continues through the vessel through the center lumen (arrows) while a perfusion lumen can be used to deliver drug to the perfusion chamber (and by extension to branches off the outer chamber). As illustrated in Panel B, following delivery, the stent can be removed by advancement of a sheath to collapse the stent. Panel C illustrates a completed stentgraft and Panel D illustrates a sheathed stent.

FIG. 2 illustrates the use of a stentgraft to deliver active agents to the spinal cord. As shown in Panel A, anatomically, arterial branches from the aorta perfuse the spinal cord (side view). The stent can be positioned over branches of a specific region of the spinal cord. As shown in Panel B, the dual chamber stent can isolate one or more spinal cord branches (here between T8 and T11) to focus drug delivery to the spinal cord. After drug delivery (a histamine portal of the blood brain barrier followed by administration of a tracer/active agent), the stentgraft can be removed by advancement of a sheath to collapse the stent.

FIGS. 3A-3C illustrate preliminary results from a proof-of-principle study demonstrating the successful delivery of a tracer (fluorsescein dye) to the spinal cord. FIGS. 3A and 3B show an angiogram of the stent with isolated drug delivery from the front (FIG. 3A) and the side (FIG. 3B) showing the perfusion chamber (outer chamber) infusing vessels of the spinal cord. In this study, a dual chamber stentgraft was used to deliver a histamine pulse followed by the tracer fluorescein to the outer chamber. As shown in FIG. 3C, when viewed under a “UV Woods lamp,” spinal cord cross sections glow from successful staining of the entirety of the spinal cord.

FIG. 4 schematically illustrates the strategy for surgically accessing and deploying the stent to allow for drug delivery to the spinal cord.

FIG. 5 illustrates the results of a proof-of-principle study in which a dumbbell-shaped dual chamber stentgraft was used to deliver a fluorescent tracer (fluorescein dye) to the spinal cord (with and without the portal agent histamine). As shown in FIG. 5, delivery of fluorescein to the spinal cord was much higher when delivered via local administration in combination with portal agent (e.g., histamine) as compared to intravascular tracer injection alone.

FIG. 6 shows the results of a proof-of-principle study evaluating the ability of the methods described herein to deliver biomacromolecules across the BBB. In this study, local administration of an antibody (a relatively large biomolecule) in combination with portal agent (e.g., histamine) was evaluated. The left panel shows a region of the spinal cord outside the drug delivery zone, showing no significant staining (i.e., no delivery of the antibody stain). Conversely, as shown in the right panel, the spinal cord within the drug delivery zone reveals significant staining with a antibody to Myelin basic protein, confirming successful delivery of a large whole antibody across the blood brain barrier with the aid of histamine

FIG. 7 shows a side view of an example stent that can be used to locally administer a portal agent and an active agent to a region of aortic branches that perfuse the spinal cord.

FIG. 8 is a cross-section view of the example stent of FIG. 7 taken along line 14-14 of FIG. 7.

FIG. 9 is a cross-section view of the example stent of FIG. 7 taken along line 15-15 of FIG. 7.

FIG. 10 is a cross-section view of the example stent of FIG. 7 taken along line 16-16 of FIG. 7.

FIG. 11 is a side view of an annular frame of an example stent, according to one embodiment.

FIG. 12A is a side view of an exemplary device of the stent of FIG. 7

FIG. 12B is a side view of the device of FIG. 12A with an integrated guidewire and nosecone.

FIG. 12C is a detail view of a portion of the device of FIG. 12B.

FIG. 12D is a side view of the guidewire and nosecone of FIG. 12B, extending from a sheath with the device of FIG. 12A and 12B radially compressed therein.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Provided herein are methods for delivering an active agent to the spinal cord of a patient. These methods can comprise locally administering a portal agent to a region of aortic branches that perfuse the spinal cord to increase permeability of the blood brain barrier; and locally administering an active agent to the region of aortic branches that perfuse the spinal cord.

In some embodiments, the portal agent and the active agent can be administered simultaneously. Alternatively, the portal agent and the active agent are administered sequentially. For example, the portal agent can be administered prior to the active agent. In certain embodiments, the active agent is administered within 30 minutes, within 15 minutes, within 10 minutes, or within five minutes of conclusion of administration of the portal agent.

The portal agent can comprise any suitable agent that disrupts the BBB and facilitates passage of an active agent. Examples of suitable portal agents include histamine, bradykinin, arachidonic acid, an eicosanoid such as a leukotriene, or any combination thereof. In certain embodiments, the portal agent can comprise histamine.

The active agent can comprise any agent that can provide a therapeutic, diagnostic, or prophylactic agent. In some embodiments, the active agent can exhibit a low permeability across the blood brain barrier in the absence of the portal agent. In certain embodiments, when locally administered in combination with the portal agent, a therapeutically effective amount of the active agent is delivered across the blood brain barrier and to the spinal cord.

In some examples, the active agent can comprise a small molecule active agent. In other examples, the active agent can comprise a macromolecule or biopharmaceutical, such as a peptide, protein, antibody, RNA, asRNA, siRNA, DNA, cDNA, or viral vector. In some examples, the active agent can comprise a stem cell. In certain embodiments, the active agent can comprise low-molecular-weight compound, a peptide (a physiologically active peptide, a hormone-like peptide, a cytokine-like peptide, a cyclic peptide, a synthetic peptide, or the like), a protein (an antibody, an enzyme, a nutritional factor, a cytokine, a hormone, or the like), a nucleic acid (a plasmid DNA, a siRNA, an miRNA, an antisense nucleic acid, a shRNA, a pre-miRNA, a pri-miRNA, an mRNA, a decoy nucleic acid, a ribozyme, a DNA aptamer, an RNA aptamer, a DNA enzyme, or the like), or a lipid

In certain cases, active agent can comprise an active agent used to treat a central nervous system disease, such as a lysosomal storage disorder, primary and metastatic cancer of the brain or spinal cord, a neurodegenerative disorder, stroke, multiple sclerosis, a CNS infection, or a traumatic injury of the brain or spinal cord.

In certain embodiments, the active agent can comprise an agent used to treat a spinal cord disease. The term “spinal cord disease” refers to a disease caused by an injury or dysfunction of the spinal cord. Examples of the spinal cord disease include amyotrophic lateral sclerosis (ALS), neuropathic chronic pain, spinal cord injury, spinal muscular atrophy, spinocerebellar degeneration, spinobulbar muscular atrophy, primary lateral sclerosis, and spinal cord tumors.

Examples of active agents for treating ALS include an active oxygen scavenger, a CYP1A2 inhibitor, an immunosuppressant, an anti-inflammatory drug, a PGE2 synthase inhibitor, an EP2 receptor inhibitor, a nutritional factor, a vitamin agent glutamate receptor antagonist, a dopamine agonist, a tyrosine kinase inhibitor, a hormone, and a nucleic acid. Specific examples include N-acetylcysteine, cyclosporine A, tacrolimus (FK506), nobiletin, a non-steroidal anti-inflammatory drug, PF-0441848, TG6-10-1, a neurotrophic factor (NGF or NT-1), a brain-derived neurotrophic factor (BDNF or NT-2), hepatocyte growth factor (HGF), vitamin 12, a vitamin B12 derivative, riluzole, perampanel, levodopa, ropinirole, bosutinib, insulin, insulin-like growth factor-1 (IGF-1), erythropoietin, and Tofersen.

Examples of active agents for treating neuropathic chronic pain include an antioxidant, a PGE2 synthase inhibitor, an EP2 receptor inhibitor, an ATP receptor inhibitor, an analgesic, an antidepressant, and an anticonvulsant. Specific examples include N-acetylcysteine, a P2X4 receptor inhibitor (5-BDBD or NP-1815-PX), PPADS, TNP-ATP, a non-steroidal anti-inflammatory drug, acetaminophen, nobiletin, an opioid, tramadol, a tricyclic antidepressant, a serotonin noradrenaline reuptake inhibitor (SNRI), a Ca²⁺ channel α6δ ligand (pregabalin, mirogabalin, or gabapentin), a Na⁺ channel inhibitory action (carbamazepine or lamotrigine), and a GABA-based activating action (sodium valproate or clonazepam).

Examples of active agents for treating a spinal cord injury include an anti-inflammatory agent, an analgesic, an active oxygen scavenger, a neurotrophic factor, a hematopoietic factor, a peptide, and a nucleic acid. Specific examples include adrenocorticosteroid, edaravone, hepatocyte growth factor (HGF), a brain-derived neurotrophic factor (BDNF), and erythropoietin.

Examples of active agents for treating spinal muscular atrophy include an antisense nucleic acid, a splicing modifier, and a siRNA. Specific examples include risdiplam and nusinersen.

Examples of active agents for treating spinocerebellar degeneration include a thyrotropin-releasing hormone (TRH) and a TRH derivative. Specific examples include mRNAs expressing Hirtonin, Ceredist, Bognin, taltirelin, protirelin, mexiletine hydrochloride, acetazolamide, and TRH.

Examples of active agents for treating spinobulbar muscular atrophy include a luteinizing hormone stimulating hormone (LHRH) analog, a heat shock protein (Hsp70) inducer, a ubiquitin-proteasome-based (UPS) activator, and a histone deacetylase (HDAC) inhibitor. Specific examples include leuprorelin, geranylgeranylacetone (GGA), and 17-allylamino-17-demethoxygeldanamycin (17-AAG).

Examples of active agents for treating primary lateral sclerosis include a muscle relaxant. Specific examples include baclofen and dantrolene.

Examples of active agents for treating a spinal cord tumor include an anticancer agent, an analgesic, an anti-inflammatory agent, an antibody, and a nucleic acid.

Local administration of the portal agent and the active agent can comprise administration to a region of aortic branches that perfuse the spinal cord such that the concentration of the portal agent and the active agent in these aortic branches is higher than the systemic, circulating concentration of the portal agent and the active agent during administration. In certain cases, local administration can comprise fluidicaly isolating these aortic branches, and administering the portal agent and the active agent into the fluidicaly isolated these aortic branches.

In certain embodimemts, locally administering the portal agent and the active agent can comprise delivering the portal agent and the active agent to a region of aortic branches that perfuse the spinal cord, wherein the region is fluidly isolated from the aorta via an implantable device. Examples of suitable devices for the local administration of a portal agent and/or an to a region of the vasculature, such as to the aortic branches to the spinal cord, include those described in U.S. Patent Application Publication No. 2023/0059358 and International Publication No. WO 2021/158377 (e.g., the artermial perfusion stents described in these references), each of which is hereby incorporated by reference in its entirety.

In some embodiments, the implantable device can comprise a first lumen configured to flow blood from an upstream end to a downstream end of the device when implanted in the aorta; and a second lumen fluidly separated from the first lumen and configured for introducing a therapeutic agent to a selected region of the aorta fluidly isolated from blood flow through the aorta, between the upstream end and the downstream end of the device where aortic branches that perfuse the spinal cord diverge from the aorta.

In some embodiments, the second lumen is arranged radially offset from the first lumen and adjacent to a central portion of the first lumen.

In some embodiments, the upstream end and downstream end of the device have a first diameter and are adapted to seal against the wall of the aorta and wherein the upstream end and downstream end are spaced apart from one another, in an axial direction, by a central portion of the device.

In some embodiments, the second lumen is arranged in the central portion of the device and the first lumen is arranged in each of the upstream end, the central portion, and the downstream end of the device.

In some embodiments, a central portion of the first lumen arranged in the central portion of the device has a second diameter that is smaller than the first diameter.

In some embodiments, the second lumen is arranged radially outside of, relative to a central longitudinal axis of the device, and surrounds at least a portion of the first lumen.

In some embodiments, the implantable device comprises a radially expandable frame including an upstream annular portion arranged at the upstream end, a downstream annular portion arranged at the downstream end, and a central portion arranged between the upstream annular portion and the downstream annular portion, the central portion including at least one narrowed portion that indents radially inward from an outermost circumference of the frame.

In some embodiments, the frame comprises a plurality of longitudinally arranged struts, relative to a direction of a central longitudinal axis of the device, which are permanently connected to converging wires which converge into a shaft of a delivery apparatus which is configured to extend outside a body of a patient.

In some embodiments, the frame comprises a plurality of longitudinally arranged struts, relative to a direction of a central longitudinal axis of the device, which converge at an end of the device into a single wire that is permanently affixed thereto.

In some embodiments, the implantable device further comprises an integrated nosecone and guidewire. In some embodiments, the integrated nosecone and guidewire can be arranged at a first end of the device and wherein the device comprises a radially expandable frame comprising a plurality of longitudinally oriented struts that converge into a single delivery wire at an opposite, second end of the device.

In some embodiments, the implantable device further comprises a perfusion lumen fluidly coupled to the second lumen and dimensioned to extend outside a body of a patient.

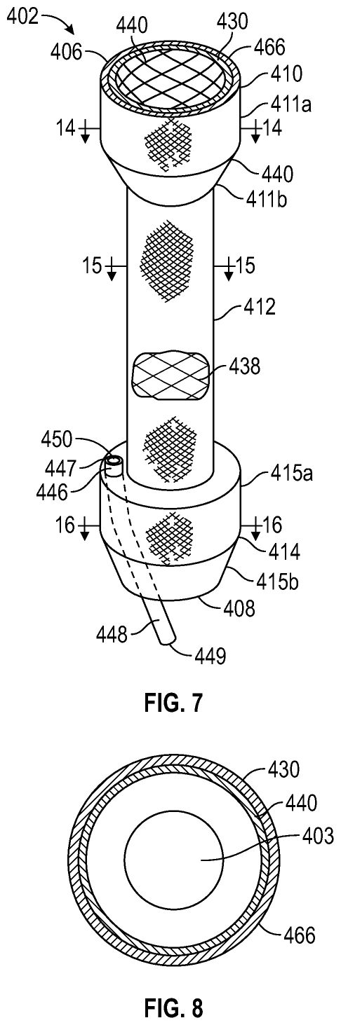

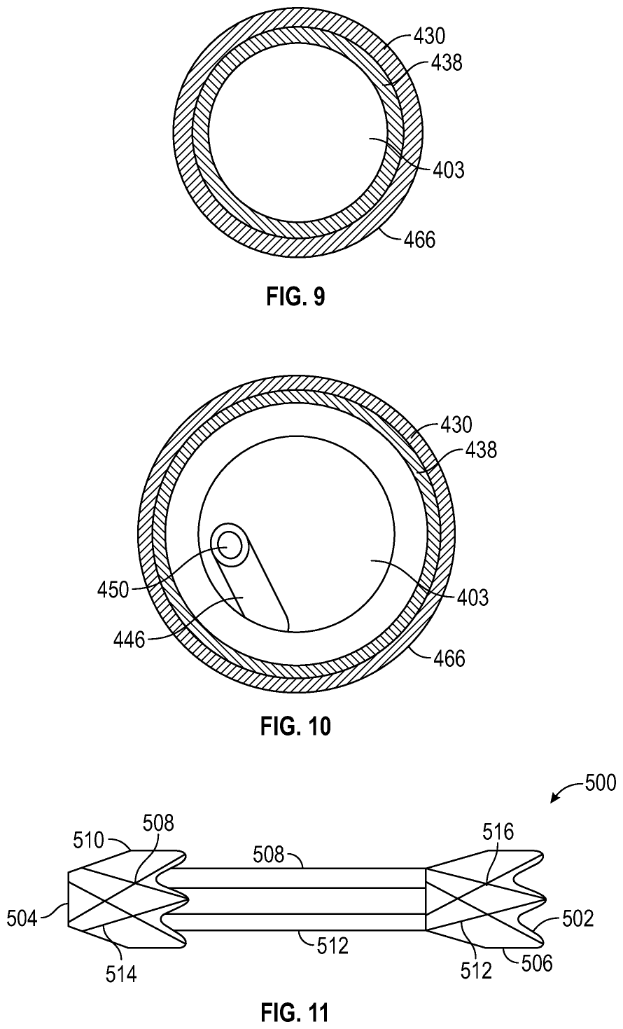

By way of example, referring to FIGS. 7-10, in some examples, the implantable device 402 comprises an elongated body that includes a radially compressible and expandable annular frame 430 supporting the liner 466. The liner can be non-porous so as to be impermeable to blood flow. The frame 430 can comprise a metal mesh, although the frame can have other configurations in other embodiments. Referring to FIGS. 7-10, in the illustrated embodiment, the stent 402 defines a central lumen 403 that extends from a proximal end 408 to a distal end 406 of the implantable device. The central lumen 403 allows passage of fluid (e.g., blood) through the body of the device, thus maintaining blood flow through the artery in which the implantable device is deployed (e.g., the aorta). The implantable device 402 can be radially compressible to a compressed state for delivery through the body to a deployment site and expandable to its functional size at the deployment site. In certain embodiments, the implantable device 402 is self-expanding; that is, the implantable device can radially expand to its functional size when advanced from the distal end of a delivery sheath. Apparatuses particularly suited for percutaneous delivery and implantation of a self-expanding implantable device in the vessels of the body are well known and described briefly below. In other embodiments, the implantable device can be a plastically-expandable device that can be adapted to be mounted in a compressed state on the balloon of a delivery catheter or another type of expansion device configured to expand the device radially from a compressed delivery state to a radially expanded state. The implantable device can be expanded to its functional size at a deployment site by inflating the balloon of a balloon catheter, as known in the art.

The elongated body of the implantable device 402 comprises a distal end portion 410, a generally cylindrical intermediate portion 412, and a proximal end portion 414. The distal end portion 410 can comprise a generally cylindrical first section 411a and a tapered second section 411b positioned proximal to the first section 411a. Likewise, the proximal end portion 414 can comprise a generally cylindrical first section 415a and a tapered second section 415b proximal to the first section 415a. In the radially expanded state of the implantable device, the distal and proximal end portions 410, 414 have an outer diameter that is larger than the outer diameter of the intermediate portion 412, thereby defining an annular perfusion space 416 between the end portions and around the intermediate portion. Central lumen 403 extends through body of the implantable device, allowing flow of fluid (e.g., blood) through from the distal end 406 to the proximal end 408 of the implantable device. The device is deployed by withdrawal of the sheath of the delivery apparatus which allows expansion of the shape memory frame as it leaves the sheath.

The outer surfaces of the distal and proximal end portions 410, 414 form a seal against the inner wall of the aorta when the implantable device is in the radially expanded state. Thus, the outer surface of the distal and proximal end portions 410, 414 of the device in the radially expanded state can have a diameter that is about the diameter of the inner surface in the region of the aorta where the stent will be placed. For example, for an implantable device to be placed in an adult, the outer surface of the distal and proximal end portions 410, 414 of the stent in the radially expanded state can have a diameter ranging from 12 mm to 3 cm. Smaller stents can be used in pediatric patients.

The device 402 can further comprise a perfusion conduit 446 that facilitates introduction of the portal agent and the active agent into the second lumen (outer chamber or annular perfusion space). The perfusion conduit 446 comprises an outlet 447 that opens into second lumen (outer chamber or annular perfusion space). The portal agent and the active agent can flow through a perfusion lumen 450. The perfusion conduit 446 can extend at least partially through the proximal end portion 414 of the body and have a proximal end that can extend beyond the proximal end portion 414, where it can be fluidly connected to a catheter 456 that extends outside of the body of the patient.

The perfusion conduit 446 can be placed anywhere in the body that allows the perfusion lumen 450 to be in fluid communication with the second lumen. In the illustrated embodiment, the perfusion conduit extends from the interior of the proximal end portion 414 of the body to the second lumen, thereby allowing such access.

FIG. 11 shows an alternate embodiment of an expandable annular frame indicated generally at 500 that can be used for the device 402. As shown, the frame 500 has a distal end 502 and a proximal end 504. The frame 500 can comprise an enlarged distal end portion 506, a generally cylindrical intermediate portion 508, and an enlarged proximal end portion 510. In the radially expanded state of the device, the distal and proximal end portions 506, 510 have an outer diameter that is larger than the outer diameter of the intermediate portion 508. The intermediate portion 508 can be formed from a plurality of longitudinally extending frame members, or struts, 512. The distal and proximal end portions 506, 510 can be formed from angled struts 514 that are welded or otherwise secured to each other at nodes 516 formed from the vertices of adjacent bends so as to form a mesh structure. Converging wires or stmts at the distal end of the stent are permanently affixed to the stent so as to allow retrieval of the stent by sheath advancement.

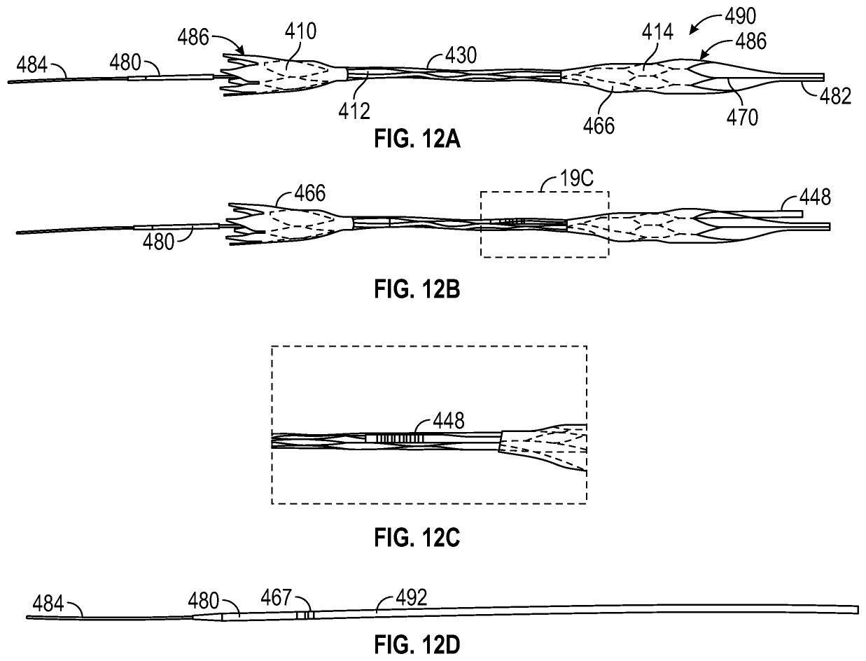

For example, converging wires 470 (as shown in FIG. 12A, which can also be referred to herein as recovery wires) may be permanently affixed to the frame of the device (e.g., at the distal end) and may converge into a single shaft or wire included as part of a delivery device. The single shaft or wire can extend outside the body of the patient and be connected, at its proximal end, to a handle or other component of the delivery device that is configured to control the delivery device (e.g., control the sheath and position of the device). In any of the implantable device embodiments described herein, the component wires of the stent/frame can be oriented longitudinally (as shown in 12A-2B for example), which facilitates device recapture and removal from the patient's body (e.g., after therapy or delivery of a therapeutic agent, as described further herein). For example, the converging, longitudinally oriented wires of the frame create a region (e.g., rail) that a sheath can be more easily slid over to collapse the stent frame and remove the device from the blood vessel.

The struts 512, 514 of the distal, intermediate, and proximal portions of the device can be made of a suitable shape memory material, such as the nickel titanium alloy known as Nitinol, that allows the device to be compressed to a reduced diameter for delivery in a delivery apparatus and then causes the device to expand to its functional size inside the patient's body when deployed from the delivery apparatus. Specifically, the shape memory device can be compressed into a delivery sheath. Advancement of the stent from the sheath, using a distal delivery wire for example, results in expansion of the stent to the shape memory dimensions of the stent.

If the device is a balloon-expandable device that is adapted to be crimped onto an inflatable balloon of a delivery apparatus and expanded to its functional size by inflation of the balloon, the perfusion stent 402 can be made of a suitable plastically expandable material, such as stainless steel.

The distal, intermediate, and proximal portions 506, 508, 510 can be constructed as a single unit, such as by machining (e.g., laser cutting). Alternatively, the frame can be constructed of separate segments each comprising respective struts or frame members, and each segment can be welded or otherwise secured together using means known in the art. In one example, the distal, intermediate, and proximal portions 506, 508, 510 are each constructed separately and secured together.

As shown in FIG. 11, the distal end portion 506 of the frame 500 in its radially expanded state can have a cylindrical shape at its distal aspect and can gradually decrease in diameter to the diameter of the intermediate portion 508. The proximal end of the distal end portion of the frame 500 is secured to the distal end of the intermediate portion 508 of the frame 430. The intermediate portion 508 of the frame in its radially expanded state generally has a uniform cylindrical shape having a diameter that is narrower than the outermost diameter of the distal and proximal end portions 436, 440 of the frame 500. The proximal end of the intermediate portion of the frame 500 is secured to the proximal end portion 510 of the frame 500. The proximal portion 510 of the frame 500 in its radially expanded state can have a cylindrical shape at its distal aspect and can gradually decrease to a narrower diameter at its proximal end, for example, to the diameter of the intermediate portion 508. The tapered proximal sections of the distal end portion 506 and the proximal end portion 510 can facilitate re-sheathing and recapture of the device, if desired.

Although a particular shape for the frame 500 is shown in FIG. 11, any shape that allows for delivery of the device to appropriate vessel location in the patient and for formation of a seal against the inner wall of the aorta and isolation of blood flow from the aorta to the visceral arties can be used.

Referring again to FIG. 7, as noted above the implantable device 402 can include a liner 466 that is non-porous to blood. The liner can be secured to the frame 430 by any suitable means, for example an adhesive or suturing. The liner covers the frame 430 of the device and prevents or substantially reduces mixing of blood flowing through the aorta and the central lumen 403 with the portal agent and active agent administered to the outer chamber. In the illustrated embodiment, the liner is located on the outside of the frame of the device. However, the liner can be located on the device in any way that provides a non-porous barrier to blood. For example, the liner can be located on the inside of the frame of the device, or on both the outside and the inside of the device.

In several embodiments, the liner 466 can be made of any suitable bio-compatible synthetic or biological material, such as those described in U.S. Pat. No. 6,730,118, which is incorporated herein by reference. The liner 466 desirably can be substantially impermeable to aqueous solutions, such as blood or plasma. In some embodiments, the liner 466 can be a polymer or composite membrane or layer, for example, polytetrafluoroethylene (PTFE); or a woven, knit, or non-woven fabric material (e.g., a ripstop fabric) manufactured from natural and/or synthetic yams or fibers, such as woven polyester (e.g., polyethylene terephthalate, PET, such as Dacron®), or cellulose (such as cotton or linen), silk, nylon, polyolefin, carbon fiber, and/or metal fibers. In additional embodiments, the liner 466 can be made of a synthetic and/or natural material that is coated with a sealant (such as ePTFE, fluoropolymer, or gelatin (Vasutek® Gelatin Sealant, Terumo, UK); see, e.g., International Publication No. WO 2001/080918, which is incorporated by reference herein in its entirety). In more embodiments, the liner 466 can be made of a bio-synthetic materials and composites (e.g., collagen-polyester composites, Omniflow®, Bio Nova, Melbourne, AU). Other embodiments use natural tissue, including intestinal submucosa, natural blood vessels (arteries or veins, e.g., from animal sources), pericardial tissue and the like, which may be fixed (for example, using gluteraldehyde and/or formaldehyde). Other embodiments include artificial collagen or cellulose tubes.

In some embodiments, the liner 466 is manufactured from sheet stock, two edges of which are brought together, for example, overlapped and/or abutted, and sealed or closed to form a tube comprising a seam. In some embodiments, the seam is linear, for example, extending along a longitudinal axis. In other embodiments, the seam has a different shape, for example, zig-zag or helical. The edges are closed using any suitable method, for example, suturing, welding, gluing, laminating, and/or bonding. In other embodiments, the liner 466 does not comprise a seam, for example, when the tubular sealing member comprises a portion of a blood vessel, intestinal submucosa, or certain artificial tubular structures.

In additional embodiments, the liner 466 can desirably be made of an electrospun polyurethane fabric (see, e.g., Amoroso et al., Elastomeric electrospun polyurethane scaffolds: The interrelationship between fabrication conditions, fiber topology, and mechanical properties. Advanced materials. 23:106-111, 2011, which is incorporated by reference herein in its entirety). Another embodiment is direct encapsulation of the frame with another polymer (e.g. polytetrafluoroethylene) to improve adherence to the frame.

In particular embodiments, the frame 430 of the device can comprise a micro-pattered thin Nitinol film (see, e.g., W02004/028340; Chun et al., Thin film nitinol microstent for aneurysm occlusion, J. Biomechanical Engineering, 131(5):051014, 8 pages, 2009; Chun et al., Novel micro-patterning processes for thin film niti vascular devices Smart Materials and Structures, 19:105021, 2010; Chun et al., Modeling and experimental analysis of the hyperelastic thin film nitinol, Journal of Intelligent Material Systems and Structures. 22, 2045-2051, 2011; Rigberg et al., Thin-film nitinol (niti): A feasibility study for a novel aortic stent graft material Journal of vascular surgery, 50:375-380, 2009; each of which is incorporated by reference herein in its entirety). Micro fabrication techniques can be used to form a plurality of micro-openings or apertures in a thin sheet of Nitinol (about 6 mM) so as to form a thin film lattice or mesh. A layer of non-porous material, such as polyurethane or ePTFE, can be applied to and secured to the metal film to provide the liner 466.

If desired, the implantable devices described herein can include suitable positioning markers and/or sensors at convenient locations to assist in locating the proximal and distal end portions of each perfusion stent at the desired locations within the aorta. For example, each of the distal end portion 410 and proximal end portion 414 of the device 402 can include a respective positioning marker. In some embodiments, the positioning markers can be radiopaque markers that can be used to locate the position of the device during deployment in a patient by radiography. For example, an x-ray image of the device within the body of the patient can be obtained using a bed-side x-ray machine to determine the position of the device within the aorta. Certain bones or other tissue visible under x-ray can be used as landmarks to help position the device relative to the visceral arteries.

In an alternative embodiment, positioning markers can be provided on the sheath. When the device 402 is located in the sheath, one marker can be aligned with the distal end portion of the device and the other marker is aligned with the proximal end portion.

In alternative embodiments, the positioning markers can comprise passive or active emitters that can emit electromagnetic waves through the body and a corresponding detector or monitor can be used to receive the electromagnetic waves from the emitters and provide visual and/or audible feedback to a user indicating the position of the markers inside the body relative to external landmarks on the body. In particular embodiments, for example, the positioning markers can be emitters that can emit radiofrequency waves, such as radiofrequency identification (RFID) or magnetic detection tags. Further details of the use of RFID tags as positioning marks are disclosed in co-pending Application No. 61/845,896, filed Jul. 12, 2013, which is incorporated herein by reference.

The implantable devices can be secured to respective one or more converging or recovery wires. The recovery wires can be secured to the proximal end of the frame of the device and can extend proximally from the device to outside the patient's body via the arteries through which the perfusion stent was deployed. If it is desired to re-position or remove the device from the patient (e.g., following drug delivery), then tension can be applied to the recovery wires to retract the device into a sheath. Once the device is retracted into a sheath, the sheath can be withdrawn from the body. The tapered sections 411b, 415b of the end portion of the device can facilitate recapture of the stent back into the sheath.

The implantable device can be inserted into the aorta via an incision in a femoral artery in a minimally invasive manner using known techniques. Guidewires, dilators and/or introducers can be used to help introduce and advance the device through the patient's vasculature, as known in the art. The proper positioning of the device can be accomplished using x-ray or fluoroscopic guidance, for example.

Once the device is in place, the proximal and distal end portions of the device can form a seal against the inner walls of the aorta, thereby isolating blood flow from the aorta to aortic branches that perfuse the spinal cord. A perfusion cannula may be integrated to the device structure or may be delivered in a modular format to the docking site 448.

In certain embodiments, the method can comprise advancing an implantable device described herein, in a radially compressed state, to a target location in the aorta where aortic branches that perfuse the spinal cord diverge from the aorta; radially expanding the device to seal a first end portion of the device against an upstream wall of the aorta and seal a second end portion of the device against a downstream wall of the aota, wherein a central portion of the device is arranged between, in an axial direction relative to a central longitudinal axis of the device, the first end portion and the second end portion where aortic branches that perfuse the spinal cord diverge from the aorta; flowing blood through a first lumen of the device, between the first end portion and the second end portion; delivering the portal agent to a second lumen of the device while flowing blood through the first lumen, the second lumen fluidly separated from the first lumen and in fluid communication with a region of the aorta where aortic branches that perfuse the spinal cord diverge from the aorta; and delivering the active agent to t second lumen of the device while flowing blood through the first lumen, the second lumen fluidly separated from the first lumen and in fluid communication with a region of the aorta where aortic branches that perfuse the spinal cord diverge from the aorta.

Also provided herein are kits for delivering an active agent to the spinal cord of a patient. These kits can comprise a composition comprising an effective amount of a portal agent as described herein to disrupt the blood brain barrier of the patient; and an implantable device as described herein for localing administering the composition comprising the effective amount of the portal agent to a region of aortic branches that perfuse the spinal cord. In certain embodiments, the kit can further comprise a composition comprising a therapeutically effective amount of an active agent to be delivered to the spinal cord of a patient.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

The reversal of spinal cord paralysis remains one of the most elusive challenges in medicine. Potential promising cures span molecular, immunomodulatory, cellular and even genetic therapies. No matter what modality of therapeutic agent is most successful, the major challenge remains how to effectively deliver these agents to the spinal cord. Surgical drug delivery is an obvious choice but carries risks of traumatic spinal cord injury that mirror spinal anesthesia risks and include Cerebrospinal fluid (CSF) leak, CSF obstruction due to inflammation, catheter complications and infection. In fact, scholars have noted that “. . . less invasive and more efficient drug administration methods are necessary to ensure patients receive the most advanced treatments with fewer complications.” The second option is injection in the space around the spinal cord (intrathecal) yet carries risks of both injury from the needle, but also compressive injury from injection of volume into a confined space.

Intravascular delivery has some distinct advantages but at the same time formidable challenges compared to topical approaches. Clearly the vascular system perfuses the entirety of the spinal cord so offers some distinct advantages over topical delivery for drug delivery, but faces two particular challenges: First, upon injection, the agent is diluted into the entirety of the circulatory system. This in turn reduces the local concentration in the spinal cord and causes loss of these agents as they are inactivated/excreted by the liver or kidneys.

Attempts to compensate for these losses or dilution by increasing the intravascular dose increases not only the cost but also the systemic toxicity of the agent. While catheterization of an individual intercostal/lumbar branch is a potential option of localizing a drug, this is both very cumbersome and lacks a fluidic seal with the vessel to prevent systemic contamination. A second, but certainly not less important barrier is the blood brain barrier (BBB), which protects the brain and spinal cord from circulating insults. For the purpose of drug delivery of larger therapies, the BBB is a formidable barrier to drug delivery to the spinal cord. In summary, while intravascular delivery offers some distinct advantages for drug delivery, an approach is needed to regionalize the drug delivery to the spinal cord, both to reduce the amount of drug delivered which in turn impacts cost and systemic toxicity.

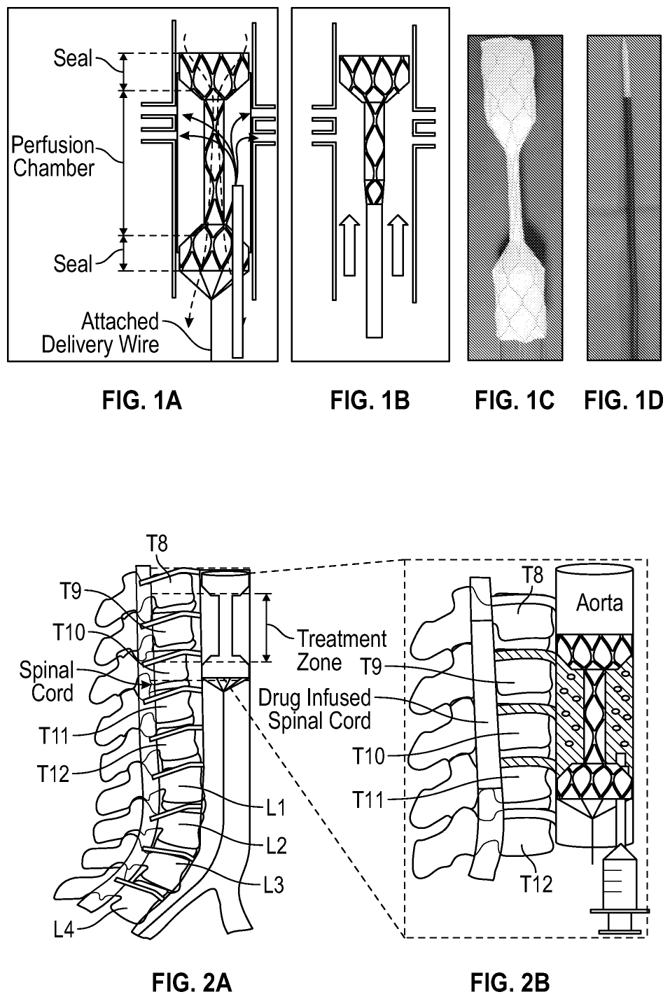

An approach is needed to temporarily breach the BBB to allow drug entry into the spinal cord tissue. We have developed retrievable stentgraft initially intended for emergent control of torso hemorrhage control while preserving distal and spinal cord perfusion. This device is a dumbbell-shaped dual chamber stent. The proximal and distal ends form a seal against the vascular wall, with preserved distal flow through the narrow center lumen (see FIG. 1, panel A). The region between the center lumen and the aortic wall is termed the “outer chamber” and is a static space isolated from the aortic flow. We have demonstrated proof of concept both by using the dual chambered stent to augment visceral flow and by the delivery of agents that induce the formation of aneurysms specifically to the wall encompassed by the outer chamber of the stent. Such a dual chamber stent can be used isolate a region of aortic branches to the spinal cord, allowing a smaller dose of therapeutic to be delivered to the spinal cord while minimizing systemic contamination.

With regards to the blood brain barrier, there are agents that are known to create “gaps” in the BBB to allow passage of larger molecular, among which histamine is the most potent. Histamine itself has systemic effects such as increased vascular permeability, allergy type symptoms and GI distress which would favor a small dose delivered directly to the target. In the context of the dual chamber stent described above, a small pulse of histamine delivered to the outer stent chamber can open the blood brain barrier, as a prelude to the therapeutic of interest yet represent a very small dose for the body overall (FIG. 2).

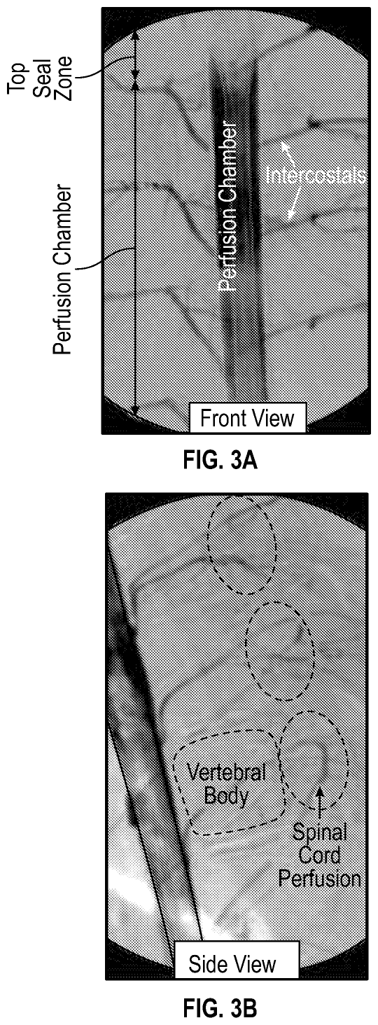

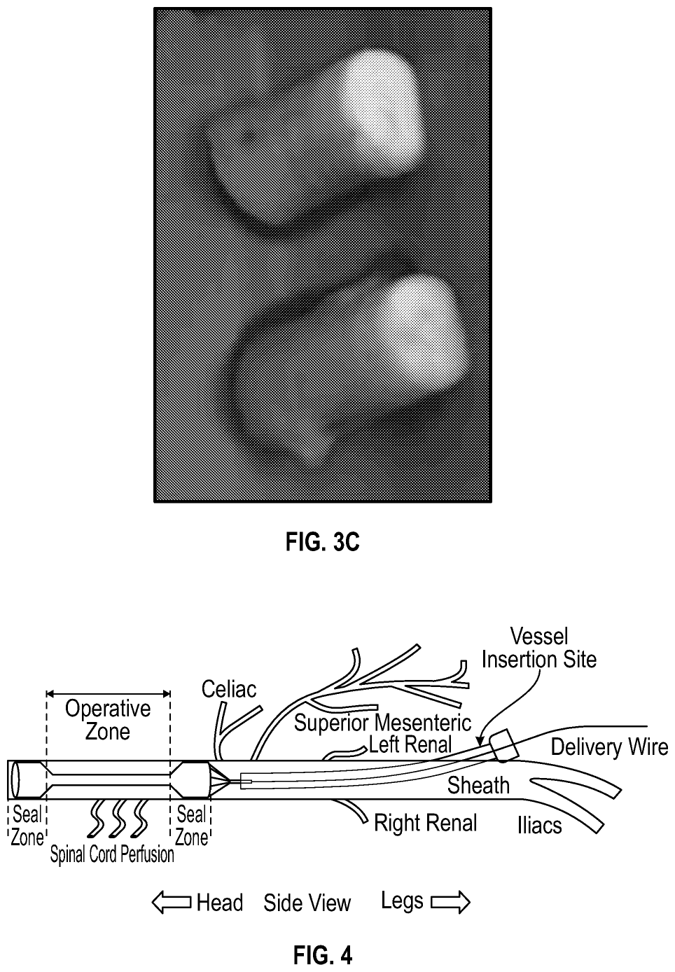

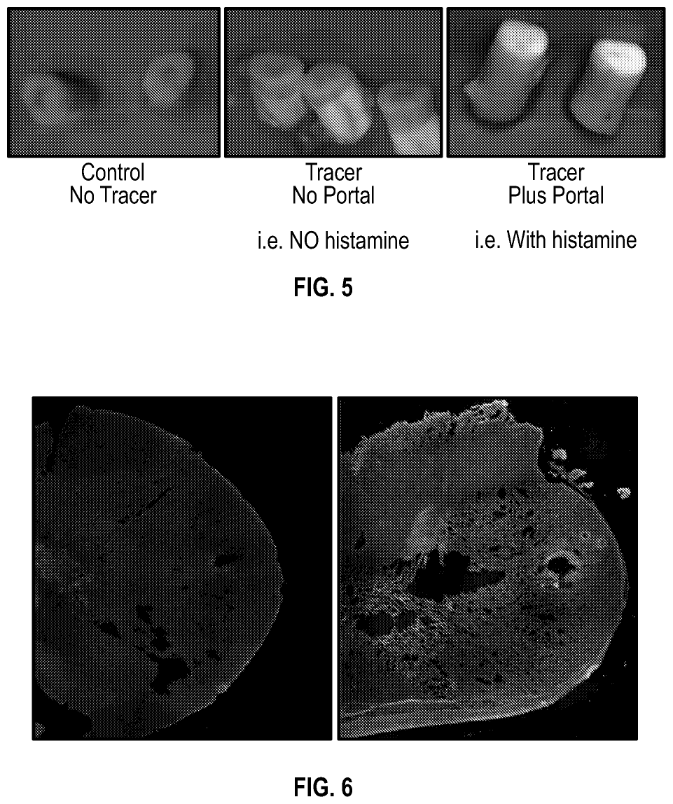

As a proof-of-principle study, we evaluated this strategy by using a dumbbell-shaped dual chamber stentgraft to deliver a fluorescent tracer (fluorescein dye) to the spinal cord. In this fluorescence tracer study, delivery of the tracer to the spinal cord using the dumbbell stentgraft was performed using a histamine portal (see FIGS. 3A-3C and FIG. 5). The dual chamber stent was used to deliver a “pulse” of histamine specifically to the spinal cord region of interest to create a “portal” in the BBB (i.e., a local increase in BBB permeability). As shown in FIG. 5, delivery of fluorescein to the spinal cord was much higher when delivered via local administration in combination with a portal agent (e.g., histamine) as compared to intravascular tracer injection alone.

FIG. 6 shows the results of an analogous proof-of principle study using an antibody stain (a stain specific to myelin basic protein) in place of fluorescein. As shown in FIG. 6, local administration of an antibody (a relatively large biomolecule) in combination with portal agent (e.g., histamine) provided for successful delivery of the antibody across the BBB and to the spinal cord. This proof-of-principle result suggests that this approach can be used to deliver large/high molecular weight active agents, such as antibodies, the spinal cord.

In certain embodiments, this strategy can be used to deliver therapies to treat spinal cord injury.

### Materials and Methods

Dual chamber, retrievable stent graft: Stents were laser cut from the shape memory alloy, nitinol, and thermally shapeset into a dumbbell shape. The scaffold was affixed to a permanently attached delivery/retrieval wire, encapsulated in the polymer polytetrafluoroethelene (Zeus, Inc) with a perfusion cannula to the outer chamber and compressed into a sheath for endovascular delivery.

Animal Model: For future studies, as part of an IACUC approved protocol, a porcine model under anesthesia will undergo iliac cannulation and heparinization. We have predicted our study will be adequately powered with six animals per each of four groups (stent +/− histamine and control aortic injection +/− histamine) to assay drug distribution.

Stent placement: The stent can be positioned fluoroscopically in the thoracic aorta and sheath withdrawal can deploy the self expanding stentgraft. To ensure continued oxygenated perfusion of the outer chamber during drug delivery, a small arterial cannula can be placed in the contralateral femoral artery. A circuit will connect the arterial cannula to the perfusion cannula of the outer chamber with a side port to allow injection of the tracer.

Pulsed BBB portal (PBP): After stent positioning over several levels of intercostal/lumbar arteries (confirmed angiographically), histamine at doses between 1-5 μg/kg will be infused into the outer chamber followed by infusion of the fluorescein tracer (14) at 3 mg/kg.

Assay of tracer distribution: Among stent animals, the optimized histamine will be infused into the outer chamber followed by the tracer. After drug delivery is complete, remaining drug is aspirated and the stent is recaptured by advancement of the sheath forward over the fixed delivery wire. Control animals will have aortic tracer injected without a stentgraft.

After the tracer circulates for 30 minutes, the animal with be euthanized and biopsies will be explanted from the liver, kidneys and heart, as well as the spinal cord recovered using a laminectomy approach. Recovered spinal cord specimens will be perfusion formalin fixed and frozen for sectioning. The fluorescein tracer will be compared by quantitating the fluorescent signal microscopically as well as within specimens of blood and urine (spectrophotometer). We will compare fluorescein tracer distribution between the study groups.

The devices, systems, kits, and methods of the appended claims are not limited in scope by the specific devices, systems, kits, and methods described herein, which are intended as illustrations of a few aspects of the claims. Any devices, systems, kits, and methods that are functionally equivalent are intended to fall within the scope of the claims. Various modifications of the devices, systems, kits, and methods in addition to those shown and described herein are intended to fall within the scope of the appended claims. Further, while only certain representative devices, systems, kits, components, and methods steps disclosed herein are specifically described, other combinations of the devices, systems, kits, components, and methods steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A method for delivering an active agent to the spinal cord of a patient, the method comprising
locally administering a portal agent to a region of aortic branches that perfuse the spinal cord to increase permeability of the blood brain barrier; and
locally administering an active agent to the region of aortic branches that perfuse the spinal cord.

2. (canceled)

3. (canceled)

4. The method of claim 3, wherein the portal agent is administered prior to the active agent.

5. (canceled)

6. The method of claim 1, wherein the portal agent is chosen from histamine, bradykinin, arachidonic acid, an eicosanoid such as a leukotriene, or any combination thereof.

7. The method of claim 1, wherein active agent exhibits low permeability across the blood brain barrier in the absence of the portal agent.

8. The method of claim 7, wherein when locally administered in combination with the portal agent, a therapeutically effective amount of the active agent is delivered across the blood brain barrier and to the spinal cord.

9. The method of claim 1, wherein the active agent comprises a small molecule active agent, a macromolecule or biopharmaceutical chosen from a peptide, protein, antibody, RNA, asRNA, siRNA, DNA, cDNA, or viral vector, a stem cell, or a combination thereof.

10. (canceled)

11. (canceled)

12. The method of claim 1, where the active agent comprises an active agent used to treat a central nervous system disease, such as a lysosomal storage disorder, primary and metastatic cancer of the brain or spinal cord, a neurodegenerative disorder, stroke, multiple sclerosis, a CNS infection, or a traumatic injury of the brain or spinal cord.

13. The method of claim 1, wherein locally administering the portal agent and locally administering the active agent comprise delivering the portal agent and the active agent to a region of aortic branches that perfuse the spinal cord, wherein the region is fluidly isolated from the aorta via an implantable device.

14. The method of claim 1, wherein the implantable device comprises:
a first lumen configured to flow blood from an upstream end to a downstream end of the device when implanted in the aorta;
a second lumen fluidly separated from the first lumen and configured for introducing a therapeutic agent to a selected region of the aorta fluidly isolated from blood flow through the aorta, between the upstream end and the downstream end of the device where aortic branches that perfuse the spinal cord diverge from the aorta.

15. The method of claim 14, wherein the second lumen is arranged radially offset from the first lumen and adjacent to a central portion of the first lumen.

16. The method of claim 14, wherein the upstream end and downstream end of the device have a first diameter and are adapted to seal against the wall of the aorta and wherein the upstream end and downstream end are spaced apart from one another, in an axial direction, by a central portion of the device.

17. The method of claim 15, wherein the second lumen is arranged in the central portion of the device and the first lumen is arranged in each of the upstream end, the central portion, and the downstream end of the device.

18. The method of claim 15, wherein a central portion of the first lumen arranged in the central portion of the device has a second diameter that is smaller than the first diameter.

19. The method of claim 14, wherein the second lumen is arranged radially outside of, relative to a central longitudinal axis of the device, and surrounds at least a portion of the first lumen.

20. The method of claim 14, wherein the implantable device comprises a radially expandable frame including an upstream annular portion arranged at the upstream end, a downstream annular portion arranged at the downstream end, and a central portion arranged between the upstream annular portion and the downstream annular portion, the central portion including at least one narrowed portion that indents radially inward from an outermost circumference of the frame.

21. (canceled)

22. (canceled)

23. (canceled)

24. The method of claim 23, wherein the integrated nosecone and guidewire are arranged at a first end of the device and wherein the device comprises a radially expandable frame comprising a plurality of longitudinally oriented struts that converge into a single delivery wire at an opposite, second end of the device.

25. The method of claim 14, further comprising a perfusion lumen fluidly coupled to the second lumen and dimensioned to extend outside a body of a patient.

26. The method of claim 1, wherein the method comprises
advancing an implantable device, in a radially compressed state, to a target location in the aorta where aortic branches that perfuse the spinal cord diverge from the aorta;
radially expanding the device to seal a first end portion of the device against an upstream wall of the aorta and seal a second end portion of the device against a downstream wall of the aota, wherein a central portion of the device is arranged between, in an axial direction relative to a central longitudinal axis of the device, the first end portion and the second end portion where aortic branches that perfuse the spinal cord diverge from the aorta;
flowing blood through a first lumen of the device, between the first end portion and the second end portion;
delivering the portal agent to a second lumen of the device while flowing blood through the first lumen, the second lumen fluidly separated from the first lumen and in fluid communication with a region of the aorta where aortic branches that perfuse the spinal cord diverge from the aorta; and
delivering the active agent to t second lumen of the device while flowing blood through the first lumen, the second lumen fluidly separated from the first lumen and in fluid communication with a region of the aorta where aortic branches that perfuse the spinal cord diverge from the aorta.

27. A kit for delivering an active agent to the spinal cord of a patient, the kit comprising:
a composition comprising an effective amount of a portal agent to disrupt the blood brain barrier of the patient; and
an implantable device for locally administering the composition comprising the effective amount of the portal agent to a region of aortic branches that perfuse the spinal cord.

28-41. (canceled)

42. The use of histamine for local administration to a region of aortic branches that perfuse the spinal cord of a patient to facilitate delivery of an active agent to the spinal cord of the patient.
